(19) United States
(12) Patent Application Publication
(10) Pub. No.: US 2018/0311468 A1
(43) Pub. Date: Nov. 1, 2018

(54) FORCE SENSOR AND MONITORING DEVICE

(71) Applicant: The Regents of the University of California, Oakland, CA (US)

(72) Inventors: Ralph V. Clayman, Irvine, CA (US); Kamaljot S. Kaler, Huntington Beach, CA (US); Jaime Landman, Irvine, CA (US); Michael J. Klopfer, Rancho Palos Verdes, CA (US); Guann-Pyng Li, Irvine, CA (US)

(21) Appl. No.: 15/963,851

(22) Filed: Apr. 26, 2018

Related U.S. Application Data
(60) Provisional application No. 62/491,011, filed on Apr. 27, 2017.

(51) Int. Cl. A61M 25/01; A61M 25/06

(52) U.S. Cl. CPC A61M 25/0105; A61M 2025/0188; A61M 2025/0681; A61M 25/0668

(57) ABSTRACT

Provided herein is a load sensing device. In one embodiment, the device comprises a load cell adapted to receive a force, an input/output interface, and an adjustable attachment mechanism adapted to reversibly attach a surgical device. In another embodiment, the load cell detects the amount of force applied during the deployment of the surgical device in a patient and outputs a force value representative thereof via the input/output interface. Also provided herein are methods of making and using load sensing devices.

### FIELD OF THE INVENTION

The present disclosure relates to the field of medical device, and specifically surgical devices.

### BACKGROUND OF THE DISCLOSURE

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

The ureteral access sheath (UAS) was first introduced in the 1970's in order to provide an accessible conduit to the kidney for the rapid and safe repeated introduction and removal of a rigid or flexible ureteroscope. Today, UAS is used commonly during flexible ureteroscopy for stone management and to diagnose and treat other upper urinary tract diseases, such as strictures or upper urinary tract tumors. However, UAS is not universally embraced due to fear of ureteral injury, Which may result from excessive force being applied during UAS placement. Currently, there is no device that measures force during UAS deployment; essentially, the UAS is deployed blindly, and there is a risk of injury, dependent upon the operator's experience and force applied. While some devices may be present for broad force sensing, and while surgical robotic systems may have used force feedback with regard to instruments abutting tissue, there remains a need for a direct hand held system available to determine force applied during manual passage of a catheter and/or sheath deployment.

### SUMMARY OF THE DISCLOSURE

Various embodiments disclosed herein include a load sensing device, comprising: a load cell adapted to receive a force; an input/output interface; and an adjustable attachment mechanism adapted to reversibly attach a surgical device; wherein the load cell detects the amount of applied force during the deployment of the surgical device in a patient and outputs a force value representative thereof via the input/output interface in a real time continuous mode. In one embodiment, the force monitoring and force output is done continuously. In one embodiment, the device further comprises software adapted to continuously collect and store the force value. In one embodiment, the data collection and storage is done wirelessly. In one embodiment, the data collection and storage is done by Bluetooth technology. In one embodiment, the adjustable attachment mechanism is a screw port. In one embodiment, the adjustable attachment mechanism is a mechanical screw port. In one embodiment, the surgical device is a device used in an endoscopic, laparoscopic, robotic, and/or minimally invasive surgical procedure. In one embodiment, the surgical device is an ureteral access sheath. In one embodiment, the input/output interface is disposable. In one embodiment, a user is capable of measuring input force during the deployment of the surgical device in a patient. In one embodiment, the device is a handheld device. In one embodiment, the device further comprises indicators to alert the user as the input force increases. In one embodiment, the indicator is a visual indicator, comprising flashing green, yellow and red lights. In one embodiment, the indicator is an audio indicator, comprising increasing sound or disharmonious sound as the force increases.

Various embodiments disclosed herein further include a method of making a load sensing device, comprising: (a) providing a load cell, an input/output interface, and an adjustable attachment mechanism adapted to reversibly attach a surgical device; (b) connecting the load cell to the adjustable attachment mechanism such that the load cell receives and monitors the amount of applied force during the deployment of the surgical device in a patient; and (c) connecting the load cell to the input/output interface such that the input/output interface outputs a force value representative of the amount of applied force. In one embodiment, the force monitoring and force output is done continuously. In one embodiment, the device further comprises providing a software adapted to continuously collect and store the force value. In one embodiment, the data collection and storage is done wirelessly. In one embodiment, the data collection and storage is done by Bluetooth technology. In one embodiment, the adjustable attachment mechanism is a screw port. In one embodiment, the adjustable attachment mechanism is a mechanical screw port. In one embodiment, the surgical device is a device used in an endoscopic, laparoscopic, robotic, and/or minimally invasive surgical procedure. In one embodiment, the surgical device is an ureteral access sheath. In one embodiment, the input/output interface is disposable. In one embodiment, the device is a handheld device. In one embodiment, the method further comprises indicators to alert the user as the input force increases. In one embodiment, the indicator is a visual indicator, comprising flashing green, yellow and red lights. In one embodiment, the indicator is an audio indicator, comprising increasing or disharmonious sound as the force increases. In one embodiment, sterile procedures are used throughout the manufacturing process.

Other embodiments disclosed herein include a method of using a load sensing device during a surgical procedure of a patient comprising: (a) providing a load sensing device comprising a load cell adapted to receive a force; an input/output interface; and an adjustable attachment mechanism adapted to reversibly attach a surgical device; (b) providing a medical device to be inserted in the patient, wherein the force sensor is not introduced at anytime into the patient; (c) attaching the load sensing device to the medical device; and (d) inserting the medical device in the patient, wherein the load sensing device monitors and outputs the force value during the medical device insertion. In one embodiment, sterile procedures are used during the use process. In one embodiment, the medical device is a device used in an endoscopic, laparoscopic, robotic, and/or minimally invasive surgical procedure. In accordance with various embodiments herein, the device would be sterile as it would be used in the operating room environment. In one embodiment, the medical device is an ureteral access sheath. In one embodiment, the load sensing device is a handheld device. In one embodiment, the load sensing device further comprises indicators to alert. the user as the input force increases. In one embodiment, the indicator is a visual indicator, comprising flashing green, yellow and red lights. In one embodiment, the indicator is an audio indicator, comprising increasing and/or disharmonious sound as the force increases. In another embodiment, at no point is the load sensing device inserted in the patient. In another embodiment, information from the load cell is incorporated into a simple disposable device. In another embodiment, the simple disposable device is intrinsic to the medical device. In another embodiment, the simple disposable device is extrinsic to the medical device. In another embodiment, the user is not able to exert more than 8 N of force when passing a ureteral access sheath. Other features and advantages of the invention will become apparent from the following detailed description, taken in conjunction with the accompanying drawings, which illustrate, by way of example, various embodiments of the invention.

### DESCRIPTION OF THE DRAWINGS

Exemplary embodiments are illustrated in referenced figures. It is intended that the embodiments and figures disclosed herein are to be considered illustrative rather than restrictive.

FIG. 1 depicts, in accordance with embodiments herein, ureteral access sheath force sensing device and screenshot of software. (A) prototype design; (B) top view; (C) side view; (D) device in view; (E) screenshot of real-time reading force sensor.

FIG. 2 depicts, in accordance with embodiments herein, force measurements of first pass ureteral dilators and UAS deployments in the right porcine ureter as measured over deployment time.

FIG. 3 depicts, in accordance with embodiments herein, force measurements of first pass ureteral dilators and UAS deployments in the left porcine ureter as measured over deployment time.

FIG. 4 depicts, in accordance with embodiments herein, mean application force during ureteral dilator and access sheath deployment.

FIG. 5 depicts, in accordance with embodiments herein, difference in mean application force between first and second pass of ureteral dilator or UAS.

FIG. 6 depicts, in accordance with embodiments herein, peak application force during ureteral dilator and access sheath deployment.

FIG. 7 depicts, in accordance with embodiments herein, change in peak measured force between first and second passage of ureteral dilator or UAS.

FIG. 8 depicts, in accordance with embodiments herein, research results utilizing UAS device. (A) medial view of normal urothelium of right proximal ureter; (B) medial wall injury of right proximal ureter and corresponding fluoroscopic image after 13F UAS placement; (C) lateral wall injury of right proximal ureter and corresponding fluoroscopic image after 14F UAS placement.

FIG. 9 depicts, in accordance with embodiments herein, UAS force sensing device including an example of a design.

FIG. 10 depicts, in accordance with an embodiment herein, a Luer-Lok interfacing ureteral access sheath force measurement and protection device. Pictured is the following: (1) Luer-Lok female connector to connect to the male Luer-Lok connector on the stylet on the top of the uretal access sheath device; (2) floating shaft; (3) device body; (4) force gauge; (5) maximum force indicator; (6) indicator at top of the floating shaft.

FIG. 11 depicts, in accordance with an embodiment herein, a shaft-coupling embodiment of the device pictured in FIG. 10. Pictured is the following: (1) Controlled forceps arm; (2) idler forceps arm; (3) uretal access sheath shaft; (4) pivot; (5) force gauge; (6) release button,

FIG. 12 depicts, in accordance with an embodiment herein, an integrated force measurement and safety release uretal access sheath. Pictured is the following: (1) finger grip; (2) uretal access sheath device body; (3) uretal access sheath shaft; (4) high-K spring; (5) collapsible bellows; (6) force gauge; (7) slide channel; (8) magnet and ferrous ring pair.

FIG. 13 depicts, in accordance with an embodiment herein, the mechanism of the device pictured in FIG. 11. Pictured is the following: Pliable roller on shaft supported by rotational bearings—connected; (2) Pliable roller on shaft supported by rotational bearings—idler; (3) pivot; (4) force clutch—shifts coaxially as force is increased as sloped teeth un-mesh; (5) high-k spring; (6) threaded support; (7) force clutch retainer and reset button; (8) force gauge.

FIG. 14 depicts, in accordance with an embodiment herein, an example of a Ureteral Access Sheath (UAS) Force Sensor Device.

FIG. 15 depicts, in accordance with an embodiment herein, sample images of various PULS scores. Green—PULS grade 0 demonstrates a. No Lesion b-d insignificant lesions as pointed out by red arrows, b. insignificant mucosal hematoma c. insignificant mucosal edema d. insignificant mucosal moulding via a guide wire. Yellow PULS grade 1 demonstrates a. superficial mucosal lesion, b-d significant lesions as pointed out by red arrows, b. mucosal hematoma, c. mucosal edema, d. mucosal moulding via a guide wire. Red—PULS Grades 2 demonstrates (a,b) submucosal lesion and (c,d) perforation with less than 50% (partial) transection; a. Video image PULS grade 2, b. Fluoroscopy image PULS grade 2 (no extravasation of contrast media), c. video image PULS grade 3, d. fluoroscopy image PULS grade 3 (extravasation of contrast media.

FIG. 16 depicts, in accordance with an embodiment herein, illustrations of PULS grade 0-5.

FIG. 17 depicts, in accordance with an embodiment herein, an example of a force measuring device connected with the UAS.

### DETAILED DESCRIPTION

All references, publications, and patents cited herein are incorporated by reference in their entirety as though they are fully set forth. Unless defined otherwise, technical and scientific terms used herein have the same meaning as commonly understood by one of ordinary skill in the art to which this invention belongs. Homyak, et al., Introduction to Nanoscience and Nanotechnology, CRC Press (2008); Singleton et al., Dictionary of Microbiology and Molecular Biology 3rd ed., J. Wiley & Sons (New York, N.Y. 2001); March, Advanced Organic Chemistry Reactions, Mechanisms and Structure 7th ed., J. Wiley & Sons (New York, N.Y. 2013); and Sambrook and Russel, Molecular Cloning: A Laboratory Manual 4th ed., Cold. Spring Harbor Laboratory Press (Cold Spring Harbor, N.Y. 2012), provide one skilled in the art with a general guide to many of the terms used in the present application. One skilled in the art will recognize many methods and materials similar or equivalent to those described herein, which could be used in the practice of the present invention. Indeed, the present invention is in no way limited to the methods and materials described.

As disclosed herein, the inventors have developed a novel medical device that is useful during a surgery. In one embodiment, the device may be used for assisting and training surgeons by measuring force during ureteric access sheath deployment in urological surgeries, but applicable to catheter and sheath insertion for urological, laparoscopic, robotic and vascular procedures. In one embodiment, the device is a force measurement device for catheter and sheath insertion procedures.

In one embodiment, the present disclosure provides a load sensing device, comprising: a load cell adapted to receive a force, an input/output interface, and an adjustable attachment mechanism adapted to reversibly attach a surgical device, wherein the load cell detects the amount of applied force during the deployment of the surgical device in a patient and outputs a force value representative thereof via the input/output interface. In one embodiment, the force monitoring and force output is done continuously. In one embodiment, the device further comprises software adapted to continuously collect and store the force value. In one embodiment, the data collection and storage is done wirelessly. In one embodiment, the data collection and storage is done by Bluetooth technology. In one embodiment, the adjustable attachment mechanism is a screw port. In one embodiment, the adjustable attachment mechanism is a mechanical screw port. In one embodiment, the surgical device is a device used in an endoscopic, laparoscopic, robotic, and/or minimally invasive surgical procedure. In one embodiment, the surgical device is an ureteral access sheath. In one embodiment, the input/output interface is disposable. In one embodiment, a user is capable of measuring input force during the deployment of the surgical device in a patient. In one embodiment, the device is a handheld device. In one embodiment, the device further comprises indicators to alert the user as the input force increases. In one embodiment, the indicator is a visual indicator, comprising flashing green, yellow and red lights. In one embodiment, the indicator is an audio indicator, comprising increasing and/or disharmonious sound as the force increases.

In one embodiment, provided herein is a method of making a load sensing device, comprising: (a) providing a load cell, an input/output interface, and an adjustable attachment mechanism adapted to reversibly attach a surgical device; (b) connecting the load cell to the adjustable attachment mechanism such that the load cell receives and monitors the amount of applied force during the deployment of the surgical device in a patient; and (c) connecting the load cell to the input/output interface such that the input/output interface outputs a force value representative of the amount of applied force. In one embodiment, the force monitoring and force output is done continuously. In one embodiment, the device further comprises providing a software adapted to continuously collect and store the force value. In one embodiment, the data collection and storage is done wirelessly. In one embodiment, the data collection and storage is done by Bluetooth technology. In one embodiment, the adjustable attachment mechanism is a screw port. In one embodiment, the adjustable attachment mechanism is a mechanical screw port. In one embodiment, the surgical device is a device used in an endoscopic, laparoscopic, robotic, and/or minimally invasive surgical procedure. In one embodiment, the surgical device is an ureteral access sheath. In one embodiment, the input/output interface is disposable. In one embodiment, the device is a handheld device. In one embodiment, the method further comprises indicators to alert the user as the input force increases. In one embodiment, the indicator is a visual indicator, comprising flashing green, yellow and red lights. In one embodiment, the indicator is an audio indicator, comprising increasing and/or disharmonious sound as the force increases. In one embodiment, sterile procedures are used throughout the manufacturing process.

In one embodiment, provided herein is a method of using a load sensing device during a surgical procedure of a patient comprising: (a) providing a load sensing device comprising a load cell adapted to receive a force; an input/output interface; and an adjustable attachment mechanism adapted to reversibly attach a surgical device; (b) providing a medical device to be inserted in the patient; (c) attaching the load sensing device to the medical device; and (d) inserting the medical device in the patient, wherein the load sensing device monitors and outputs the force value during the medical device insertion. In one embodiment, sterile procedures are used during the use process. In one embodiment, the medical device is a device used in an endoscopic, laparoscopic, robotic, and/or minimally invasive surgical procedure. In one embodiment, the medical device is an ureteral access sheath. In one embodiment, the load sensing device is a handheld device. In one embodiment, the load sensing device further comprises indicators to alert the user as the input force increases. In one embodiment, the indicator is a visual indicator, comprising flashing green, yellow and red lights. In one embodiment, the indicator is an audio indicator, comprising increasing and/or disharmonious sound as the force increases.

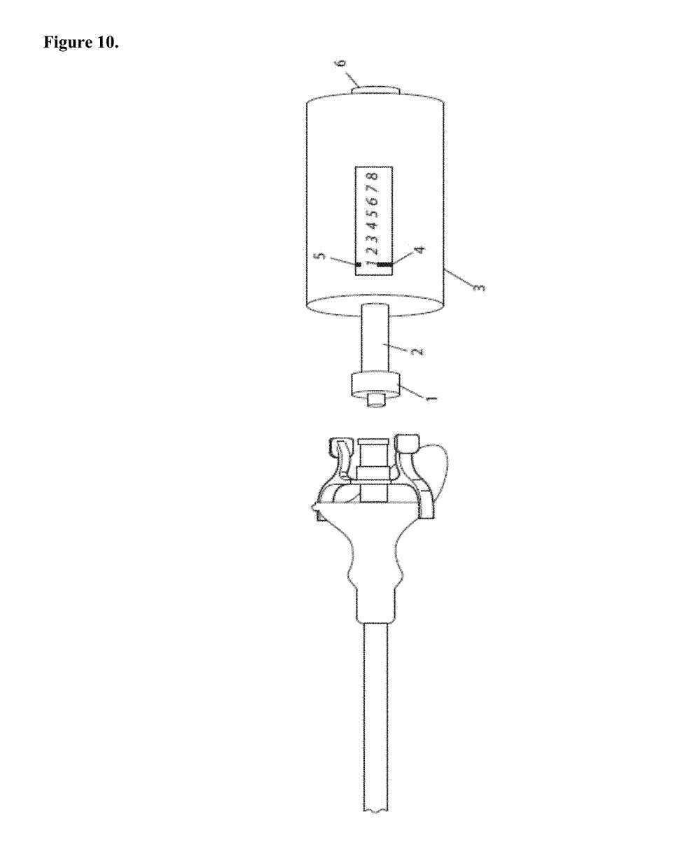

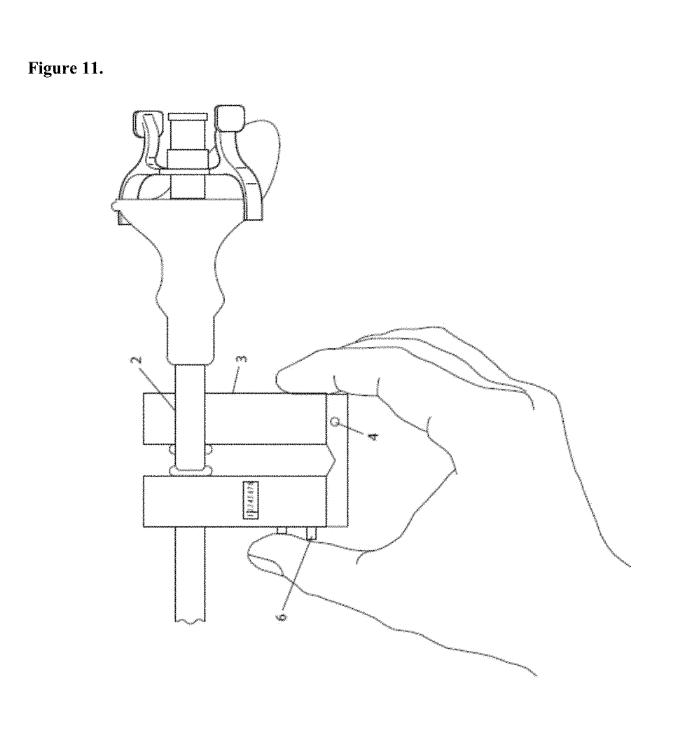

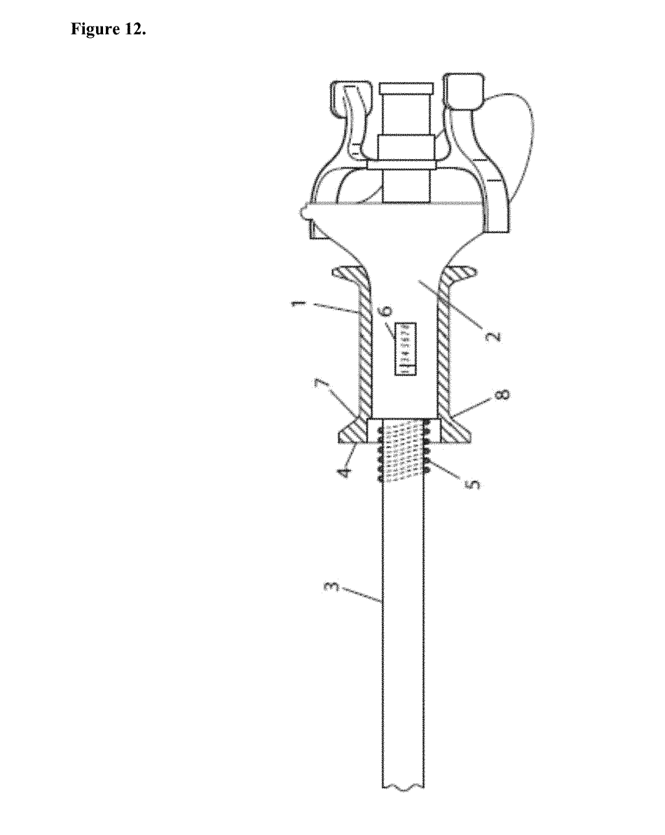

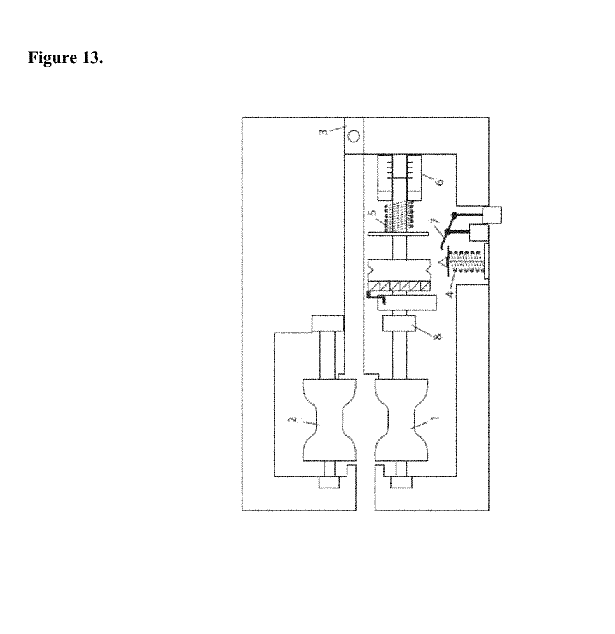

As described herein, in one embodiment, the device may be used for assisting and training surgeons by measuring force during ureteric access sheath (UAS) deployment in urological surgeries, as well as applicable to catheter and sheath insertion for urological, laparoscopic, robotic and vascular procedures. Looking at FIGS. 10-13 herein specifically, for example, one can see illustrated the device as either intrinsic with the access sheath or extrinsic as a device that can be slipped over any access sheath and used to measure force. In accordance with various embodiments herein, for example, in one embodiment, the present invention provides a device where based on the information obtained from using a load cell, one could incorporate that information into a simple disposable device, for example, that was either intrinsic to the medical device, or UAS, or extrinsic to it, such as slipped over it, such that the operator would not be able to exert more than 8 N of force, for example, when passing the UAS.

Embodiments of the present disclosure are further described in the following examples. The examples are merely illustrative and do not in any way limit the scope of the invention as claimed.

### EXAMPLES

### Example 1

Pressure monitoring during UAS insertion

In one embodiment, the present disclosure provides a force measurement device for catheter and sheath insertion procedures. In one embodiment, the device can be directed towards urological sheath insertion force measurement.

The ureteral access sheath (UAS) was first introduced in the 1970's in order to provide an accessible conduit to the kidney for the rapid and safe repeated introduction and removal of an ureteroscope. Today, UAS is used commonly during flexible URS for stone management and to diagnose and treat other upper urinary tract diseases, such as strictures or upper urinary tract tumors. However, UAS is not universally embraced due to fear of ureteral injury, which may result from excessive force being applied during UAS placement. Currently, there are no devices that measures forced during UAS deployment; essentially, the UAS is deployed blindly, and there is a risk of injury, indeed. In accordance with various embodiments herein, the device disclosed herein can be used for training and quality control/standardization purposes.

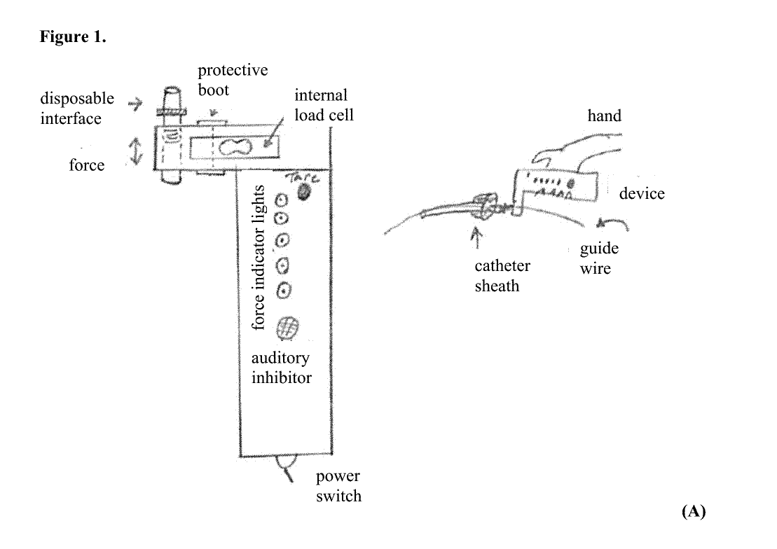

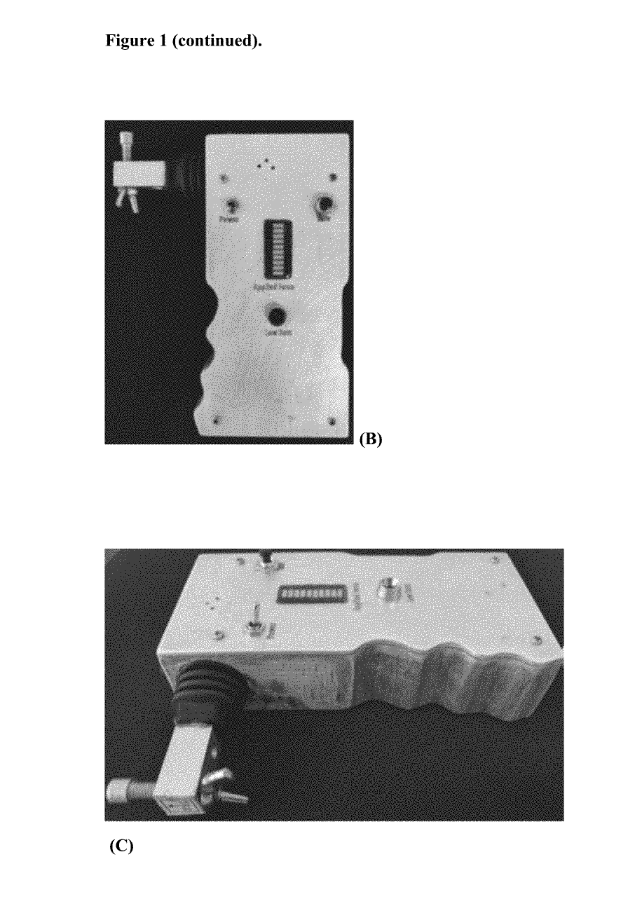

Currently there is no direct hand held system known to the inventors available in the market or discussed in the literature to help with manual catheter or insertion sheath deployment. As such, the inventors designed and developed a novel ureteral access sheath force sense device to be used in the operating room during any procedure requiring UAS insertion. FIG. 1 provides an embodiment of the device. The device will be connected to the UAS using sterile technique; for this a mechanical interface port (screw-port for a luer-lock connector in the current design) housed in a sterile clear plastic bag is secured to the UAS. The device uses a load cell (based on multiple strain gauges) to measure force and would provide continuous measurements during deployment of the UAS. The device reports ad-hoc to the surgeon via visual and audio indicators of applied force. The hand held device component of the system links to an Android based tablet with a custom application a custom display and control application that interfaces with the device using Bluetooth technology that is connected to an external computer (running Android operating system) that will display the force in real time.

In one embodiment, as illustrated in FIG. 1 herein, the device is in a form factor to allow measurement of catheter sheath insertions via a side mounted load cell. In another embodiment, the device is shaped to provide easy and ergonomic use in surgery. In one embodiment, the device is all metal and hermetically sealed providing water resistance for sanitization procedures and easy gas sterilization. In one embodiment, the device interfaces via Bluetooth technology to a tablet based application to provide a GUI with controls. In one embodiment, the Bluetooth antenna is internally mounted near the rubber gasket to provide sufficient range while contained within an all metal case construction.

In one embodiment, clinical studies were performed that for the first time define a safe range of force during ureteric access sheath deployment and define a range of excessive force that causes ureteral damage. Data from these experiments would further justify device usage but also should help to prevent injuries during surgical sheath insertion. In one embodiment, a sheath is developed with a purposely built-in failure mode such that use of excessive force results in buckling of the sheath distally and thus failure of the proximal end of the sheath to advance thereby shielding the ureter from exposure to excessive force. In one embodiment, the device is also contemplated to be used in procedures such as laparoscopy, robotics, vascular and interventional radiology procedures.

### Example 2

Pressure sensing device

Ureteral injuries have been noted to occur during ureteral access sheath (UAS) deployment. The three exerted during deployment has yet to be measured; likewise the amount of force that results in ureteral injury has not yet been quantitated. The inventors have developed a novel force-sensing device and tested its feasibility to identify, for the first time, the threshold force that reliably induces a ureteral injury in a porcine model.

With IACUC approval, ureteral instrumentation (ureteral dilator and UAS) deployment force was measured using the Ureteral Access Sheath Force Sensor (UAS-FS) device disclosed herein. The force exerted was measured continuously from when the tip of the access sheath was introduced at the urethral meatus to when the distal end of the dilator was hubbed at the urethral meatus; insertion of the sheath was monitored fluoroscopically. Each catheter/sheath (6-16 F) was passed twice. Ureteroscopic evaluation was performed with each catheter/sheath after the 9.5 F obturator was passed in order to look for potential ureteral injuries. Injuries were graded using the Post-Ureteroscopic Lesion Scale (PULS, from 0—no injury to 5 severe ureteral disruption).

No injuries were detected when the deployment force remained under 4 Newtons (N) which was the case when the UAS was ≤13 F. Increasing UAS size above 13 F resulted in larger force measures during UAS passage and greater peak forces. First ureteral injury was seen with a peak force of 8 N and 10 N in the right and left ureters, respectively in the initial porcine study.

The UAS-FS can measure force applied to a catheter or sheath in a reproducible fashion. Peak force <4 N was found to be safe during UAS deployment in a porcine model. One-time peak force ≥8 N resulted in ureteric injury. A clinical study proposal has been approved by the UC Irvine IRB and is now in progress.

### Example 3

Generally

Flexible ureteroscopy (URS) has become a method of definitive management for small ureteral and upper urinary tract calculi. The ureteral access sheath (UAS) was developed to facilitate the repeated passage of the flexible ureteroscope. Use of the UAS is associated with decreased intrarenal pressures, improved stone free rate, and reduced operative time, and a reduction in ureteroscope repair costs.

Although the UAS has undergone design modifications to improve usability (e.g. size variety, reductions in buckling and kinking, and a hydrophilic coating) its use is not yet widespread. Stress forces exerted upon the ureteral wall during UAS deployment have raised concerns. A prospective investigation by Traxer et al estimated the incidence of UAS-induced ureteral injury to be as high as 48%, with serious injuries (i.e. ureteral perforation/splitting) noted in 13% of clinical cases. An established threshold of application force and a method of real-time force measurement during UAS deployment could markedly improve the safety of using UAS during URS.

In an effort to characterize the forces exerted upon the ureteral wall during UAS deployment, the inventors have developed a novel load-sensing device, the UCI Ureteral Access Sheath Force Sensor (UAS-FS). In one embodiment, the UAS-FS device disclosed herein would allow an user to measure the applied forces, identify a safe range of forces, and define a threshold force beyond which UAS-induced damage would be most likely to occur.

### Example 4

Methods

Ureteral Access Sheath Force Measuring Device Development: The Ureteral Access Sheath Force Sensor (FIG. 1) device was designed for sterile use in the operating room during endoscopic procedures requiring placement of a UAS. An internal load cell is used as a strain gauge to measure input force during UAS deployment. The device is equipped with a disposable interface, in which a mechanical screw-port (i.e. Luer-Lok™ mechanism) can be secured to the UAS using sterile technique. Bluetooth technology and device specific software allow for continuous wireless data collection (i.e. pound-force (lbf) or gram-force (gf)) via an external wireless device (such as Android device). All UAS-FS measurements were converted to Newtons (N) (1 N=0.225 lbf=101.97 gf). In addition, the device provides both real-time visual ((via force indication lights from green (low force) to yellow to red (excessive force)) and audio (increasing sound) graded feedback to alert the user as the input force increases. The device can be sterilized using a STERRAD system (Ethicon US, LLC).

Animal Preparation: Following approval by the Institutional Animal Care and Use Committee (IACUC), a 35-kilogram female Yorkshire pig was placed under general anesthesia. Vital signs, temperature, and muscle tone were continuously monitored throughout the procedure as per the Guide for the Care and Use of Laboratory Animals.

Preliminary Ureteral Access: With the animal supine, the pelvis was prepared with povidone-iodine solution and standard drapes were applied. A flexible cystoscope (11272 VP, Karl Storz Inc., Germany) was introduced into the urethra and a 0.035 in. guidewire (Amplatz Super Stiff Guidewire, Boston Scientific Corp., Natick, Mass.) was placed into the left ureteral orifice and fluoroscopically guided to the kidney. Once studies on the left ureter were completed, the procedure was repeated on the right ureter.

Ureteral Dilator and Access Sheath Deployment Force Measurement and Assessment of Ureteral Damage: A single individual (KK) performed all ureteral dilator and UAS deployments over the guidewire using the UAS-FS. Force measurement was initiated upon insertion of the UAS tip at the urethral meatus and stopped when the UAS was either hubbed at the meatus or the tip of the device had reached the renal pelvis; the entire procedure was done under fluoroscopic guidance. Measurements were recorded using several ureteral dilators (6 F, 7.5 F, 8 F, 9 F) and UAS obturators and their respective sheath (9.5/11 F, 35 cm; 10/12 F, 35 cm; 11/13 F, 36 cm; 12/14 F, 35 cm; 13/15 F, 36 cm; 14/16 F, 35 cm) (Cook Medical® Inc., Bloomington, Ind. & Boston Scientific Corp., Natick, Mass.). Forces were recorded twice (i.e. Pass 1 and Pass 2) for both the right and left ureter, respectively. After the passage of the 9.5 F obturator, flexible ureteroscopy (Flex-X2, Karl Storz Inc., Germany) was performed for each subsequent passage of larger dilators/obturators/sheaths to assess for ureteral injury. The appearance of an ureteral injury was recorded and the location confirmed with fluoroscopy. Injuries were graded using the Post-Ureteroscopic Lesion Scale (PULS) on a scale of “0” no injury to “5” severe ureteral disruption. The porcine bladder was emptied between deployments using a 10 F Foley catheter.

### Example 4

Results and analysis

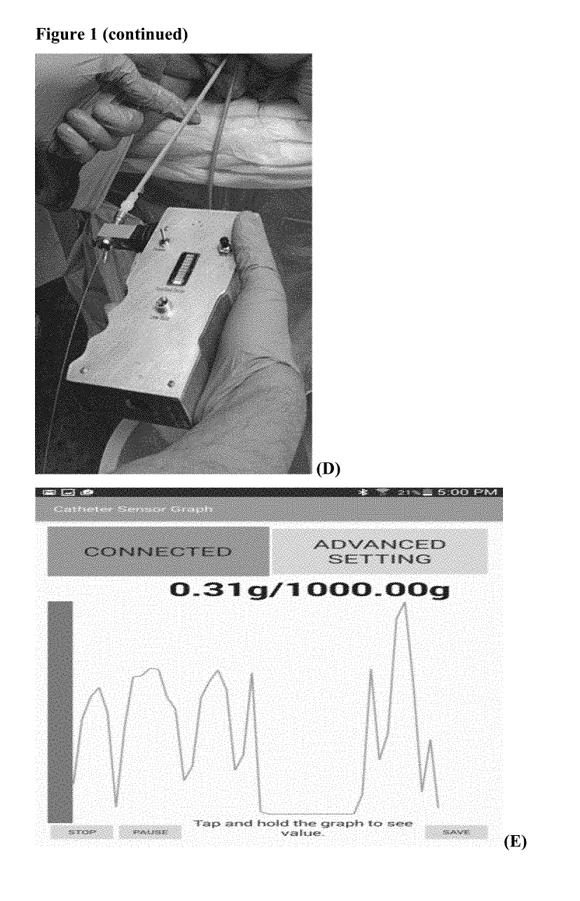

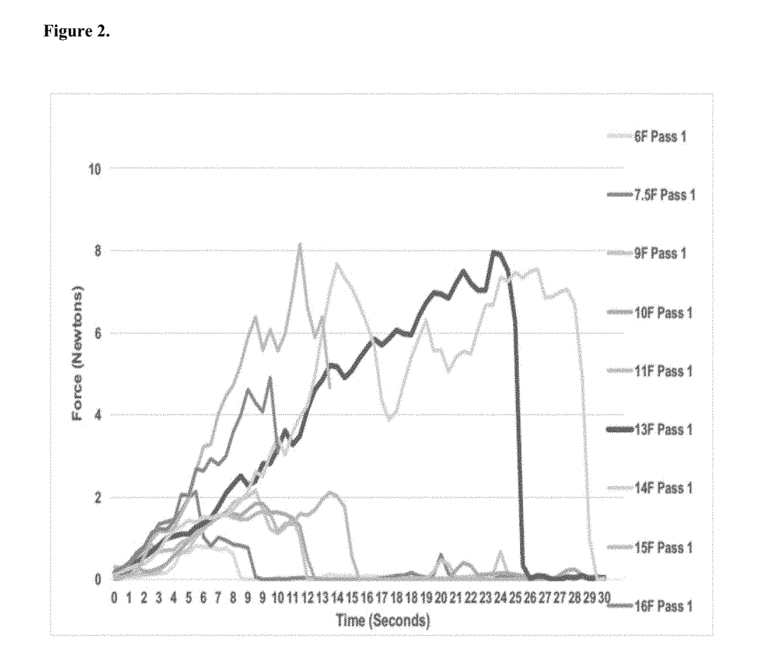

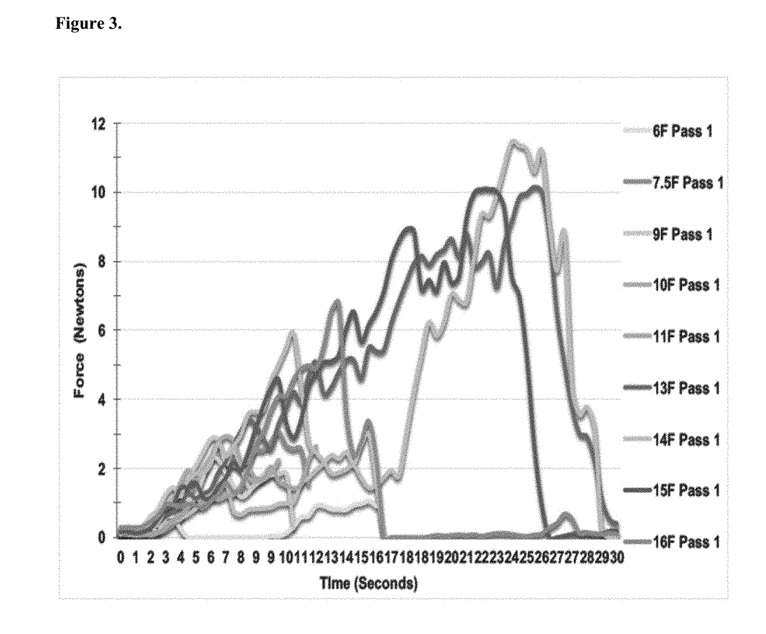

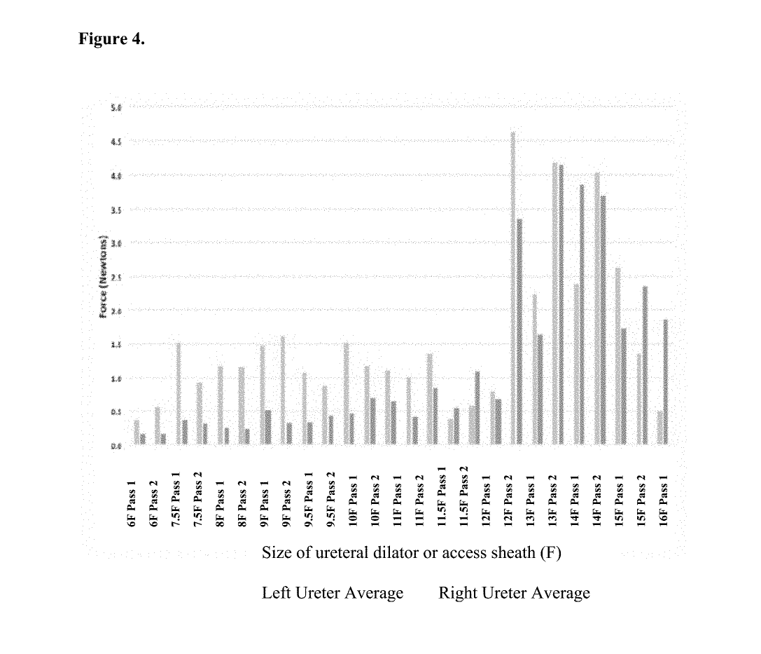

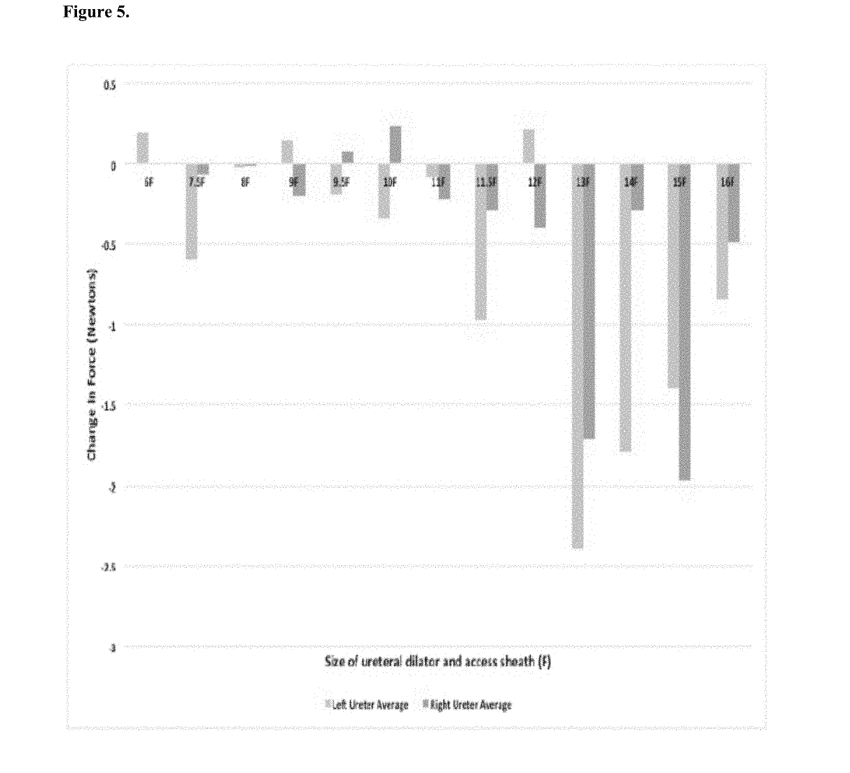

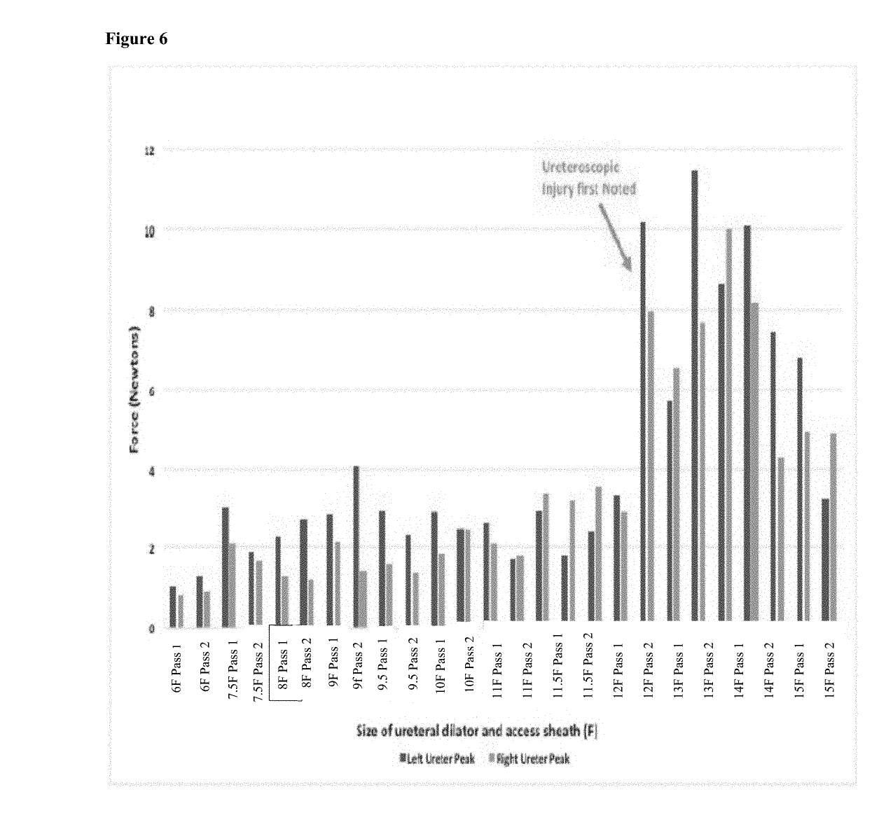

In one embodiment, force measurements were performed during ureteral dilator and access sheath deployment. The measured deployment force increased with larger diameter UAS in both the right and left ureter (FIG. 2 & FIG. 3, respectively). The mean application force for each insertion remained relatively steady until deployment of the 13 F UAS (FIG. 4). The mean application force of the second pass was often lower than the first (FIG. 5). Both the mean and peak application force notably increased following placement of the 13 F UAS (FIG. 6). The greatest change in mean application force (>1 N) between the passes was seen with UAS ≥13 F. Similarly, the peak application force was notably higher during deployment of UAS, 11.5 F.

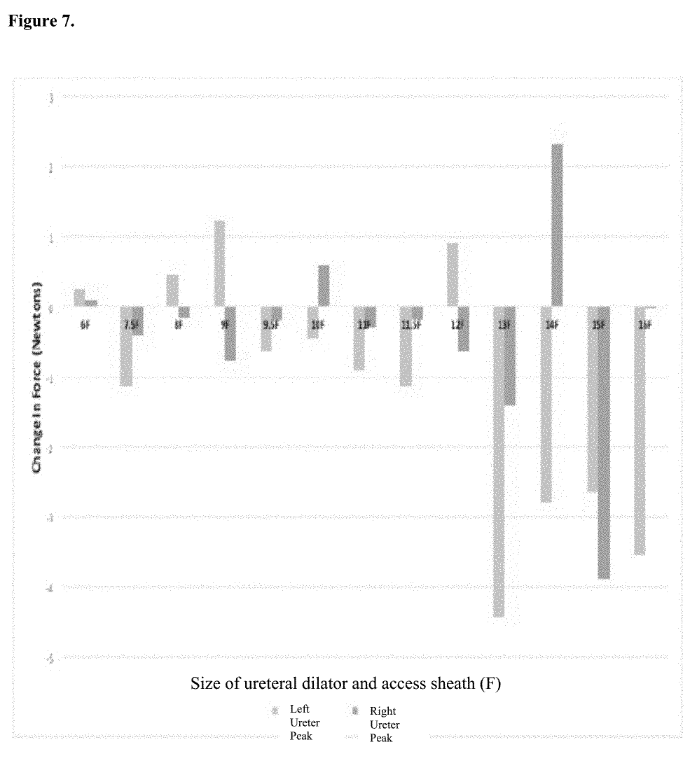

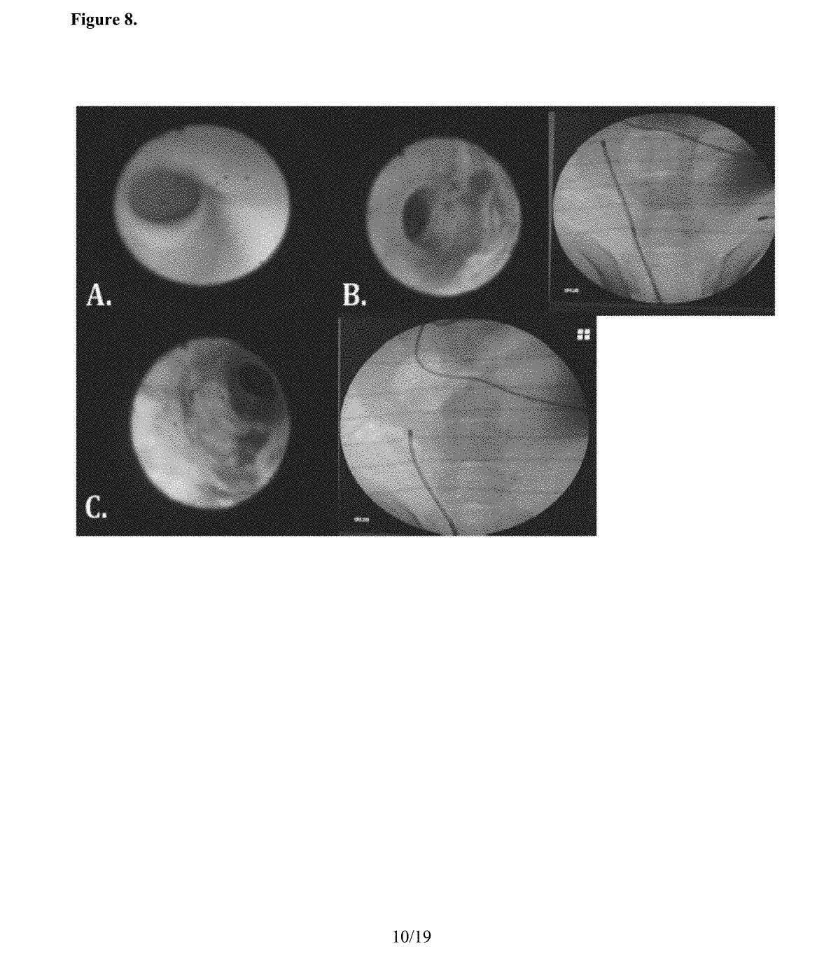

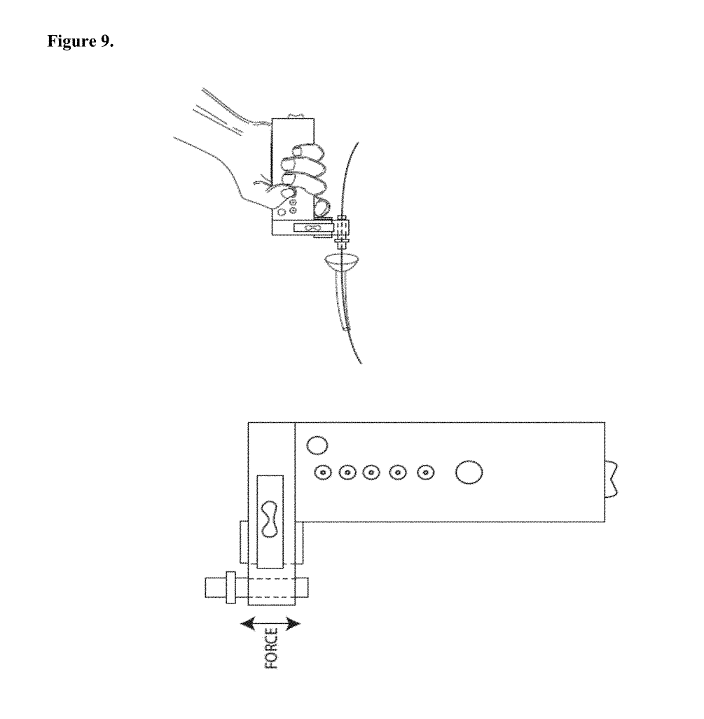

Ureteral injuries were noted during placement of the 13 F UAS, with a peak force of 8 N on the right ureter and 10 N on the left (FIG. 5 & FIG. 7). These injuries were both graded as a PULS 3. Subsequent PULS 4 injury to the right ureter occurred following placement of the 14 F UAS. Although a peak force of 4.8 N was recorded, further injury (PULS 4) to the right ureter was visualized during deployment of the 16 F UAS, likely due to the fact that the urothelium had already been split and the integrity of the ureteral wall once breached, resulted in further spreading of the edges with minimal force applied. Images and corresponding fluoroscopic location of ureteral injuries are demonstrated in FIG. 8.

Generally, the ureteral access sheath has faced scrutiny since its introduction largely due to urothelial injury and potential concerns over subsequent ureteral stricture formation. Concerns were reported in the literature with the original UAS upon discovering a ureteral perforation in 8 of 43 (19%) cases in a prospective study (Newman et al, 1987). Numerous design changes occurred over the following decade (e.g. tapered proximal tip of the inner dilator, metal coiling of the outer sheath to preclude kinking, and a liquid-activated hydrophilic coating) to decrease the risk of ureteral injury. Nevertheless, in a prospective investigation of 359 patients undergoing UAS-facilitated URS, low-grade ureteral injury was reported in 86% of patients and high-grade injury in 13% of patients. (Traxer et al). The force required to induce such injuries has not been defined and thus one is left with only an anecdotal, empirical sense of how hard to push on the UAS in order to position it.

In this seminal study, the inventors aimed to define how much force could be applied to the ureteral wall before it would split. To this end, the inventors have developed a novel load-sensing device (UAS-FS) that could be used in the operating room to measure the applied force throughout the deployment of a catheter or UAS over a guidewire. In this study 8 N of force resulted in injury to the adult female porcine ureter. When forces remained under 4 N no injury was observed. As the diameter of the UAS increased so did the force required to deploy it; however, it was not until a 13 F sheath was passed that 8 N was reached and a ureteral injury was noted. Not surprisingly, once the ureter was “dilated” from the first pass of the 13 F sheath, during the second pass there was less force needed to deploy the same sized UAS (FIG. 5). Of importance, once an injury had occurred, less force was required to induce further injury.

The concept of evaluating force placed on the ureter during rigid ureteroscopy (URS) was first introduced by Ulvik et al.; they created a novel coupling device to attach a standard force meter to a 8.5 F semirigid ureteroscope to measure forces exerted in the ureter during rigid URS. Mean retrograde insertion force ranged from 9.7±7.3 N at the distal ureter to 4.4±3.6 N at the proximal ureter. Although no complications were reported, the Ulvik study did not grade ureteral injury nor provide long-term patient follow-up.

Similar studies utilizing porcine models have been performed to assess the force of UAS deployment. A feasibility study by Harper et al., compared the insertion force following placement of a novel radially dilating 9.5 F and a conventional 12/14 F UAS in a porcine model to the level of the UPJ. The researchers reported mean forces of 0.48 and 2.2 N, respectively, and maximum forces of 1.6 and 6.5 N, respectively. These results are similar to the current findings using the UAS-FS during 9.5 F and 12/14 F UAS deployment (mean application force of 0.67 and 3.63 N, respectively), however peak application force showed greater variation (1.4 to 2.9 N and 7.6 to 11.4 N, respectively). Furthermore, although the Harper study used a novel trauma score to assess ureteral damage (n.b.: not PULS) following UAS placement, the amount of force at the moment of injury was not provided. Of note, like Ulvik and colleagues, they used a standard force sensor that was not designed specifically for force measurement of a catheter or sheath deployed over a guidewire.

Lidal et al. evaluated the effect of isoproterenol on the insertion force of placing a 13/15 F UAS in the porcine ureter, again using a standard force sensor with a coupling mechanism. During UAS placement, input force was measured and deployment was stopped once subjective resistance was met by the operator. Subsequently, the pharmacological intervention (i.e. isoproterenol vs, saline) via a 10 F ureteral catheter was delivered. Input force for ureters treated with isoproterenol was significantly less than those irrigated with saline. Importantly, mean endpoint values 5.34 N (pre-irrigation) and 4.6 N (in 6 ureters with no subjective resistance) are within limits of mean (1.7-4.0) and peak (4.2-10.0) 15 F UAS forces measured in this instant study with the UAS-FS. Their study corroborated that the force applied, and resulting ureteral injury, varied with the operator thus highlighting the importance of being able to actively measure application force during UAS deployment in real time. Their study did not measure forces for different sized LAS or define a threshold for injury.

The study and results disclosed herein provide reliable continuous measurements in a clinical setting. In one embodiment, the device disclosed herein may also provide a threshold level for injury; however, at this point in time those observations remain merely anecdotal. A standard force transducer was not used for comparison with our novel UAS-FS because conventional force transducers are not specifically designed to be used over a guidewire.

The novel UCI UAS-FS accurately and continuously measures forces during ureteral dilator or LAS deployment in a reproducible manner in an adult female pig. A peak force of less than 4 N was not associated with any ureteral injuries; however, a single, even brief application of 8 N force was sufficient to cause an ureteral injury.

### Example 5

Impact of force applied during UAS deployment on Ureteral injury in a Porcine Model

Widespread use of the ureteral access sheath (UAS) during ureteroscopy has been slowed by concerns over possible ureteral injury during its passage. In this porcine study, using the novel device disclosed herein, the inventors evaluated the force threshold which would induce ureteral injury.

The inventors measured UAS deployment force using a novel Ureteral Access Sheath Force Sensor (UC Irvine Force Sensor) in a female Yorkshire pig. (FIG. 1). Under fluoroscopic control, force was continuously measured from the time the UAS contacted the urethral meatus until the tip of the UAS had reached the renal pelvis. Ureteral dilators (6-9 F), UAS and its obturators (9.5 F, 10 F, 11 F, 11.5 F, 12 F, 13 F, 14 F, 15 F, 16 F) sequentially passed twice into both ureters. Ureteroscopic evaluation was initiated after the 9.5 F UAS obturator was passed. No ureteral injury occurred at ≤4 Newtons (N). Increasing UAS size resulted in greater force over-time and larger peak forces (FIG. 4). First ureteral injury occurred at 8 N (right ureter) and 10 N (left ureter). FIG. 8 shows a normal right ureter before and after deployment of a 13 F LAS with a peak force of 8 N. Accordingly, the force sensor device disclosed herein can reliably and continuously measure force while deploying a UAS. Initial ureteral injury occurred at forces >8 N.

### Example 6

UAS Force Device Experiment Summary

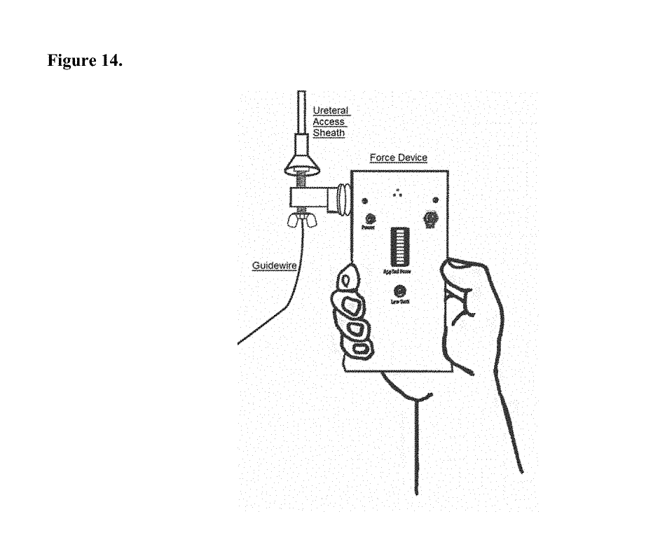

Concerns regarding ureteral injury have limited the more widespread use of an ureteral access sheath (UAS) during ureteroscopy. The amount of force during ureteral access sheath deployment that results in clinical ureteral injury has not been defined. Two experiments were performed using the ureteral access sheath force sensor device (FIG. 14). The inventors performed a follow-up porcine study with more extensive testing of the novel UAS Force Sensor (UAS-FS). They also continued clinical testing of the sensor during routine ureteroscopy and percutaneous nephrolithotomy in stone patients.

### Porcine Model Experiment:

The UAS-FS was used to measure UAS deployment forces in six female Yorkshire pigs (average size 19.5 kg (16-22 kg). Under fluoroscopic guidance, ureteral dilators (6-9 F) along with a variety of UAS and corresponding obturators (9.5 F-16 F) were sequentially advanced into one of the ureters in each pig. In the other ureter, after 8/10 F dilation, the 12/14 F UAS was deployed without any sequential dilation. Force was measured continuously from the time the UAS entered the urethral meatus until the tip of the UAS reached the renal pelvis. The bladder was drained between deployments as needed. Ureteroscopic evaluation was performed after each dilation to assess for ureteral injury using the post ureteroscopic lesion scale (PULS)≥3.

No PULS 3 ureteral injury was observed at forces 5.5 Newtons (N). Increasing UAS diameter resulted in larger peak forces. In 4/6 pigs, ureters selected for 12/14 UAS deployment without prior sequential dilation were injured. Ureters subjected to sequential dilation did not incur a significant injury (i.e. PULS 3) until a peak force of 8.83 N was reached. The UASFS reliably measure force while deploying a UAS. Initial ureteral injury within these smaller pigs was avoided provided the peak force remained <5.5 N.

### Clinical Experiment:

In the initial porcine study, the inventors noted that a peak force of 8 Newtons during ureteral access sheath deployment resulted in splitting of the ureter. With this information, the inventors began clinical testing using UAS-FS (FIG. 14) during routine ureteroscopy and percutaneous nephrolithotomy in stone patients.

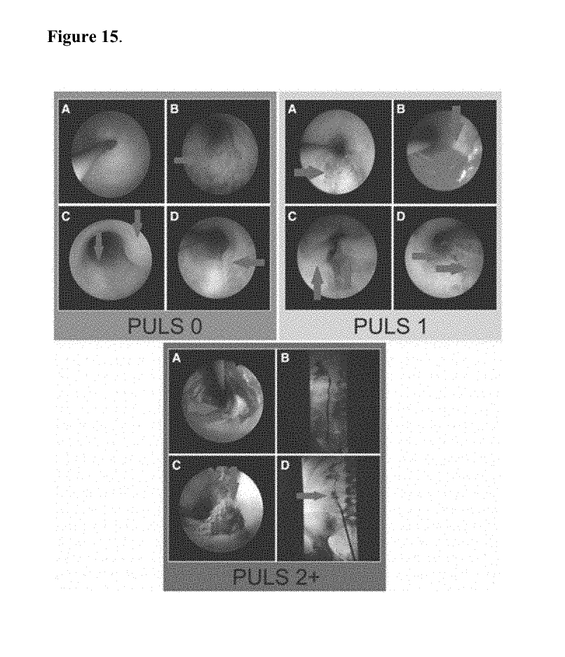

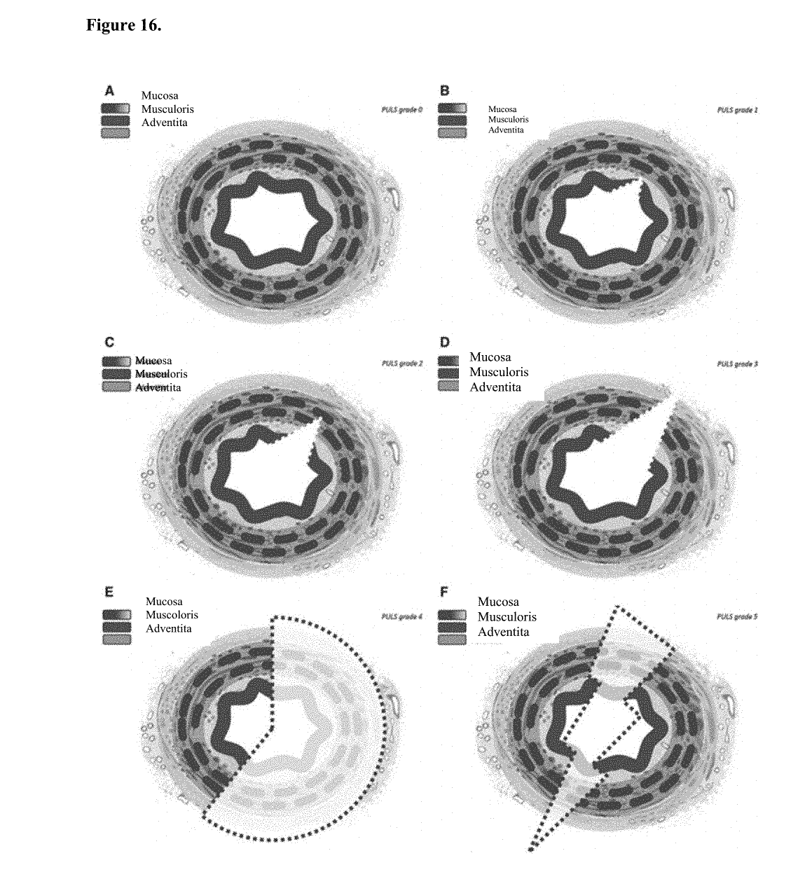

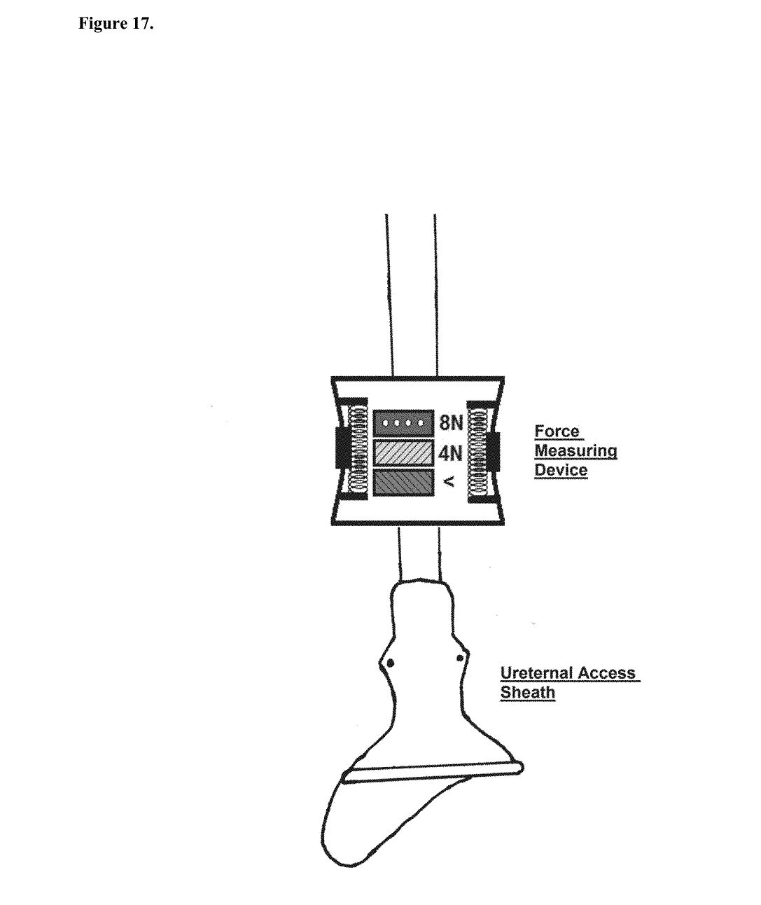

UAS deployment force was measured in 35 patients using UAS-FS under fluoroscopic control by 4 different surgeons. Tamsulosin was given for up to one week in two-thirds of these patients to induce a state of ureteral relaxation. Continuous UAS-FS measurements began from when the tip of the UAS was inserted into the urethra until the tip of the UAS reached the ureteropelvic junction. If the force approached/began to exceed 8 N (audible sound), passage was stopped, progress of the UAS was recorded fluoroscopically, and the UAS was withdrawn and a smaller UAS selected. Ureteroscopic evaluation of the entire ureter was performed at the end of each case to assess for potential ureteral injuries using the post-ureteroscopic lesions scale (PULS) (Table 1; FIGS. 15-16 herein).

Among the 35 patients, there were 41 UAS deployments. The 16 French UAS could be passed at <8 N in 61% of patients; in the remainder, a smaller UAS was deployed (14 F in 13 cases and 11.5F in 2 cases) being careful to not exceed 8 N. The mid ureter location was where the maximum peak pressure (20%) was most commonly recorded. The mean PULS grade was 0.77. The solitary PULS 3 injury occurred in a patient in whom three UAS insertions were serially tried 16 F, 14 F, and 11.5 F at peak forces of 8.1 N. 8.9 N and 5.0 N, respectively; Of note, in the other 34 patients, peak recorded pressure never exceeded 8 N. The UAS-FS was able to measure UAS insertion force in a reproducible fashion. Limiting the force exerted on the UAS to <8 N, precluded a PULS score over 2 in all patients.

### Example 7

TABLE 1
Post Ureteroscopic Lesion Scale (Grade 0-5)

| | | |
|---|---|---|
| Grade 0 | No lesion | Uncomplicated URS |
| Grade 1 | Superficial mucosal lesion | (no grading according to the |
| | and/or significant | Dindo-modified Clavien |
| | mucosal edema/ | classification of surgical |
| | hematoma | complications) |
| Grade 2 | Submucosal lesion | |
| Grade 3 | Perforation with less than | Complicated URS |
| | 50% partial transsection | |
| Grade 4 | More than 50% partial | (Grade 3a or b according to |
| | transsection | the Dindo-modified Clavien |
| Grade 5 | Complete transsection | classification of surgical |
| | | complications) |

The various methods and techniques described (vide supra) provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps, sonic will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the selection of constituent modules for the inventive compositions, and the diseases and other clinical conditions that may be diagnosed, prognosis made too, or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a,” “an,” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein.

Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

What is claimed is:

1. A load sensing device, comprising:
a load cell adapted to receive a force;
an input/output interface; and
an adjustable attachment mechanism adapted to reversibly attach a surgical device,
wherein the load cell detects the amount of force applied during the deployment of the surgical device in a patient and outputs a force value representative thereof through the input/output interface.

2. The load sensing device of claim 1, wherein the force monitoring and force output is done continuously.

3. The load sensing device of claim 1, further comprising software adapted to continuously collect and store the force value.

4. The load sensing device of claim 3, wherein the data collection and storage is done wirelessly.

5. The load sensing device of claim 3, wherein the data collection and storage is done by Bluetooth technology.

6. The load sensing device of claim 1, wherein the adjustable attachment mechanism is a screw port.

7. The load sensing device of claim 1, wherein the adjustable attachment mechanism is a mechanical screw port.

8. The load sensing device of claim 1, wherein the surgical device is a device used in an endoscopic, laparoscopic, robotic, and/or minimally invasive surgical procedure.

9. The load sensing device of claim 1, wherein the surgical device is an ureteral access sheath.

10. The load sensing device of claim 1, wherein the input/output interface is disposable.

11. The load sensing device of claim 1, wherein a user is capable of continuously measuring input force in real time during the deployment of the surgical device in a patient

12. The load sensing device of claim 1, wherein the device is a handheld device.

13. The load sensing device of claim 1, wherein the device further comprises indicators to alert the user as the input force increases.

14. The load sensing device of claim 13, wherein the indicator is a visual indicator, comprising flashing green, yellow and red lights.

15. The load sensing device of claim 13, wherein the indicator is an audio indicator, comprising increasing and/or disharmonious sound as the force increases.

16. A method of making a load sensing device, comprising:
providing a load cell, an input/output interface, and an adjustable attachment mechanism adapted to reversibly attach a surgical device
connecting the load cell to the adjustable attachment mechanism such that the load cell receives and monitors the amount of force applied during the deployment of the surgical device in a patient; and
connecting the load cell to the input/output interface such that the input/output interface outputs a force value representative of the amount of force applied.

17. The method of claim 16, wherein the force monitoring and force output is done continuously.

18. The method of claim 16, further comprising providing a software adapted to continuously collect and store the force value.

19. The method of claim 18, wherein the data collection and storage is done wirelessly.

20. The method of claim 18, wherein the data collection and storage is done by Bluetooth technology.

21. The method of claim 16, wherein the adjustable attachment mechanism is a screw port.

22. The method of claim 16, wherein the adjustable attachment mechanism is a mechanical screw port.

23. The method of claim 16, wherein the surgical device is a device used in an endoscopic, laparoscopic, robotic, and/or minimally invasive surgical procedure.

24. The method of claim 16, wherein the surgical device is an ureteral access sheath.

25. The method of claim 16, wherein the input/output interface is disposable.

26. The method of claim 16, wherein the device is a handheld device.

27. The method of claim 16, further comprises indicators to alert the user as the input three increases.

28. The method of claim 27, wherein the indicator is a visual indicator, comprising flashing green, yellow and red lights.

29. The method of claim 27, wherein the indicator is an audio indicator, comprising increasing and/or disharmonious sound as the force increases.

30. The method of claim 16, wherein sterile procedures are used throughout the manufacturing process.

31. A method of using a load sensing device during a surgical procedure of a patient comprising:
providing a load sensing device comprising a load cell adapted to receive a force, an input/output interface, and an adjustable attachment mechanism adapted to reversibly attach a surgical device;
providing a medical device to be inserted in the patient;
attaching the load sensing device to the medical device; and
inserting the medical device in the patient, wherein the load sensing device monitors and outputs the force value during the medical device insertion.

32. The method of claim 31, wherein sterile procedures are used during the use process.

33. The method of claim 31, wherein the medical device is a device used in an endoscopic, laparoscopic, robotic, and/or minimally invasive surgical procedure.

34. The method of claim 31, wherein the medical device is an ureteral access sheath.

35. The method of claim 31, wherein the load sensing device is a handheld device.

36. The method of claim 31, wherein the load sensing device further comprises indicators to alert the user as the input force increases.

37. The method of claim 36, wherein the indicator is a visual indicator, comprising flashing green, yellow and red lights.

38. The method of claim 36, wherein the indicator is an audio indicator, comprising increasing and/or disharmonious sound as the force increases.

39. The method of claim 31, wherein at no point is the load sensing device inserted in the patient.

40. The method of claim 31, wherein information from the load cell is incorporated into a simple disposable device.

41. The method of claim 40, wherein the simple disposable device is intrinsic to the medical device.

42. The method of claim 40, wherein the simple disposable device is extrinsic to the medical device.

43. The method of claim 40, wherein the user is not able to exert more than 8 N of force when passing a ureteral access sheath.
